(19) United States
(12) Patent Application Publication
(10) Pub. No.: US 2024/0257063 A1
(43) Pub. Date: Aug. 1, 2024

(54) Computerized System and Method of Capturing and Reporting Physicians Time

(71) Applicant: Crowe LLP, Oak Brook, IL (US)

(72) Inventors: Andrew Jay Sutton, Zionsville, IN (US); Ronald Kenyon Wolf, JR., Wildwood, MO (US); Rebecca Jane Brugler, Warren, OH (US); Emily Jo Auten, St. Peters, MO (US); David Joseph Andrzejewski, Ballwin, MO (US); Scott Edward McNeany, Indianapolis, IN (US); Gregory Allen Becerra, Fishers, IN (US)

(21) Appl. No.: 18/633,998

(22) Filed: Apr. 12, 2024

Related U.S. Application Data
(63) Continuation of application No. 14/208,245, filed on Mar. 13, 2014, now abandoned.
(60) Provisional application No. 61/789,115, filed on Mar. 15, 2013.

(51) Int. Cl. G06Q 10/1091; G06Q 40/12; G16H 40/20; G16H 70/20; G16Z 99/00

(52) U.S. Cl. CPC G06Q 10/1091; G06Q 40/125; G16H 40/20; G16H 70/20; G16Z 99/00

(57) ABSTRACT

A computerized system and method for capturing and reporting physician time in a manner that complies with reimbursement regulations. The system eases the difficulty of collecting physician time, thereby solidifying reimbursement revenue. The system has integrated rules to ensure compliance with time allocations. Moreover, a structured work flow for review of time entries is disclosed.

### RELATED APPLICATIONS

This is a continuation of U.S. application Ser. No. 14/208,245 filed Mar. 13, 2014 for a “Computerized System and Method of Capturing and Reporting Physicians' Time,” which claimed the benefit of U.S. Provisional Application Ser. No. 61/789,115 filed Mar. 15, 2013 for a “Computerized System and Method of Capturing and Reporting Physicians' Time.” Both of these applications are hereby incorporated by reference in their entireties.

### TECHNICAL FIELD

This disclosure relates generally to computerized systems and method; in particular, this disclosure relates to a computerized system and method for capturing, documenting, allocating, and reporting physician time on cost reports.

### BACKGROUND AND SUMMARY

Many industries track time spent on projects for billing purposes. In certain fields, such as consultants and attorneys, workers track billable hours that are charged at the worker's hourly rate to a client. There are many software programs available that aid in tracking billable hours which interface with accounting systems for generating invoices with the billable time.

In some cases, physicians must also track time spent on various activities through time studies. Unlike other professionals, however, merely keeping track of time is insufficient because regulations dictate numerous rules governing the manner by which the time study must be conducted to obtain reimbursement. For example, Medicare regulations (42 CFR §§ 415.55 and 415.60) indicate that costs a provider incurs for services of physicians are allowable and subsequently reimbursable if certain conditions are met, including:
The provider submits a written allocation agreement that specifies the respective amounts of time the physician spends furnishing services to provider (e.g. administrative and teaching), services to patients, and services not payable under Medicare (e.g., research).
The provider maintains the time records it used to allocate physician compensation in a form that permits validation by the Medicare Administrative Contractor (MAC).

The criteria for use of periodic time studies are explained in further detail in the Provider Reimbursement Manual (“PRM”) instructions (CMS Pub 15-1 § 2313.2E) and includes the following:
The time records to be maintained must be specified in a written plan submitted to the MAC no later than 90 days prior to the end of the cost reporting period to which the plan applies.
A minimally acceptable time study must encompass at least one full week per month of the cost reporting period.
Each week selected must be a full work week (e.g. Mon-Fri, Mon-Sat, or Sun-Sat).
The weeks selected must be equally distributed among the months in the cost reporting period (e.g., for a 12 month period, 3 of the 12 weeks in the study must be the first week beginning in the month, 3 weeks the 2^{nd} week, etc.).
No two consecutive months may use the same week for the study (e.g., if the second week beginning in April is the study week for April, the weeks selected for March and may not be the second week beginning in those months).
The time study must be contemporaneous with the costs to be allocated. Thus, a time study conducted in the current cost reporting year may not be used to allocate the costs of prior or subsequent cost reporting years.
The time study must be provider specific. Thus, chain organizations may not use a time study from one provider to allocate the costs of another provider or a time study of a sample group of providers to allocate the costs of all providers within the chain.

With the increased demands on physician schedules, it is difficult to accurately capture, document, and report on physician time. Traditional time studies result in calculation and data entry errors and mistakes are usually not discovered until an audit, if ever at all. The entire process is unnecessarily labor-intensive, error prone, and costly. Therefore, there is a need for a system to improve the capture, documentation, allocation and reporting of physician time that complies with these regulations to ensure reimbursement.

In one aspect, this disclosure relates to a computerized system and method for capturing and reporting physician time in a manner that complies with reimbursement regulations. The system eases the difficulty of collecting physician time, thereby solidifying reimbursement revenue. Moreover, the system has integrated rules to ensure compliance with time allocations.

Additional features and advantages of the invention will become apparent to those skilled in the art upon consideration of the following detailed description of the illustrated embodiment exemplifying the best mode of carrying out the invention as presently perceived. It is intended that all such additional features and advantages be included within this description and be within the scope of the invention.

### BRIEF DESCRIPTION OF THE DRAWINGS

The present disclosure will be described hereafter with reference to the attached drawings which are given as non-limiting examples only, in which:

FIG. 1 is a diagrammatic view of a computerized system for capturing, reporting and managing physicians' time according to one embodiment of the present invention;

FIG. 2 is a block diagram showing various functions of the system according to one embodiment of the present invention;

FIG. 3 is a diagrammatic view of a contract management work flow according to one embodiment of the present invention;

FIGS. 4-24 are screenshots showing possible functionality of an administrative module according to one embodiment of the present invention;

FIG. 25 is a diagrammatic view of a time entry and review work flow according to one embodiment of the present invention; and

FIGS. 26-81 are screenshots showing possible functionality of a time entry/review module according to one embodiment of the present invention;

Corresponding reference characters indicate corresponding parts throughout the several views. The components in the figures are not necessarily to scale, emphasis instead being placed upon illustrating the principals of the invention. The exemplification set out herein illustrates embodiments of the invention, and such exemplification is not to be construed as limiting the scope of the invention in any manner.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the disclosure.

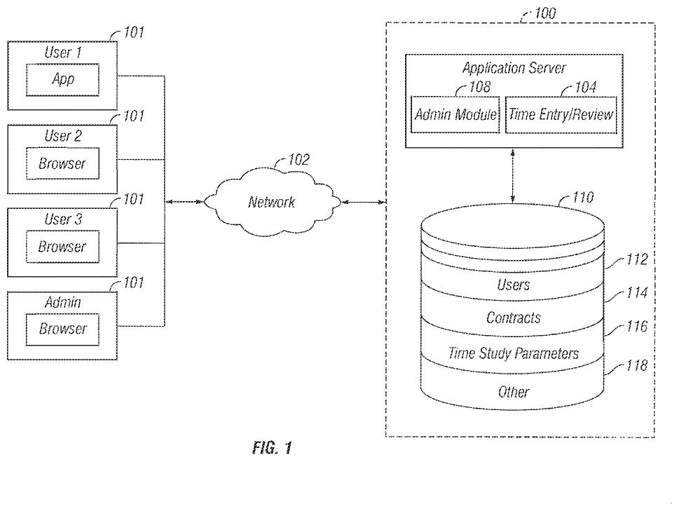

FIG. 1 is a diagrammatic representation of a server 100 that is in communication with a plurality of user machines 101 (although 4 are shown for purposes of example, one or more than one could be provided) via a network 102 to provide one or more of the functions described herein, such as an administrative module 108 and/or a time entry/review module 104. The system 100 could be accessed on the machines 101 using a webpage or using a dedicated application. For example, the machines 101 could be a tablet device or smart phone, such as an iOS™ or Android™ device, that has an “app” loaded thereon which is capable of accessing one or more functions of the administrative module 108 and/or the time entry/review module 104. In some cases, the administrative module 108 and/or time entry/review module 104 could be accessed from the server 100 via the network 102 using a browser program, such as Internet Explorer™ by Microsoft Corporation of Redmond, Washington, or Safari™ by Apple Corp. of Cupertino, California. Typically, the administrative module 108 and the time entry/review module 104 are configured to store various types of data in a database 110. In the example shown, the database 110 includes information about users 112, contracts 114, time study parameters 116, and other data 118.

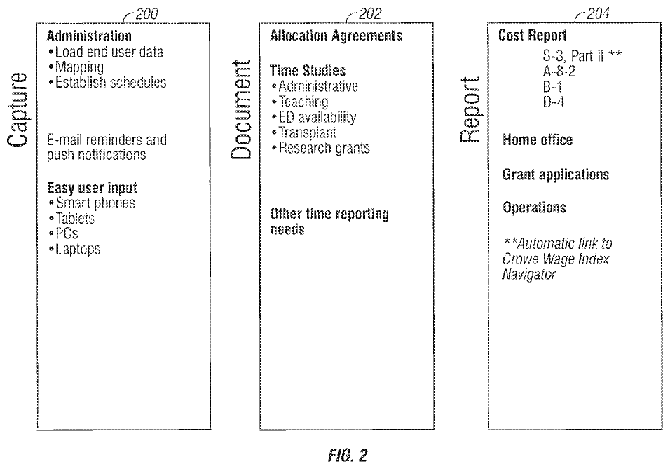

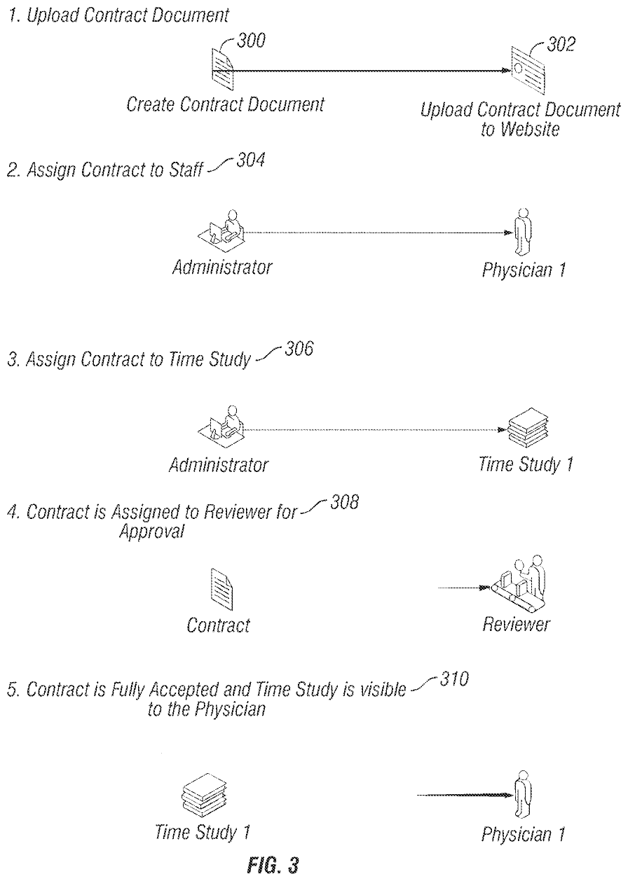

FIG. 2 is a block diagram showing various functions of the system 100. In this example, there is a capture module 200 that is configured to load in user data regarding time entries and map these to individual users and departments. This allows easy input of time entries by the user, such as using smart phones, tablets, PCs, and/or laptops. In this embodiment, the system 100 also includes a document module 202 that is used to store and manage various allocation agreements. For example, these could be time studies for administrative, teaching, ED availability, transplant, research grants, and other time. In this embodiment, the system 100 also includes a report module 204 which may be used to produce various reports from the underlying data. In some cases, the data may be exported or otherwise provided to other applications for further processing.

FIG. 3 is a diagrammatic view of a contract management workflow in one embodiment. In the example shown, a contract document may be created (block 300) and uploaded to a contract document website (block 302), where it is stored in a database. The contract may be assigned to a particular physician (block 304) by an administrator using the system. The contract may then be associated with a time study (block 306). Upon being assigned to a particular time study, the contract is assigned to a reviewer to determine whether the contract needs approval (block 308). If the contract is fully accepted, the time study becomes visible to the physician for entry of time records (block 310).

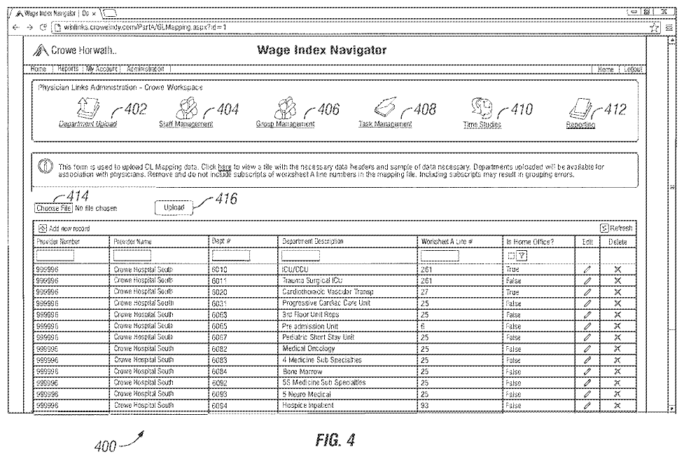

FIGS. 4-24 are screen shots showing possible functionality of an administrative module 108 that may be used to store contracts and assign these to various physicians, departments, and time studies, along with other functionality. FIG. 4 shows a home screen 400 with a plurality of categories that may be selected by a user depending on the functionality that is desired to be accessed. In the example shown, the system administrative module 108 includes the following categories that may be selected by a user: department upload 402, staff management 404, group management 406, task management 408, time studies 410, and reporting 412. In the example shown, the user has selected the department upload category 402. As shown in this example, the user may select a choose file button 414 that may be used to select a file and then upload it by selecting the upload button 416.

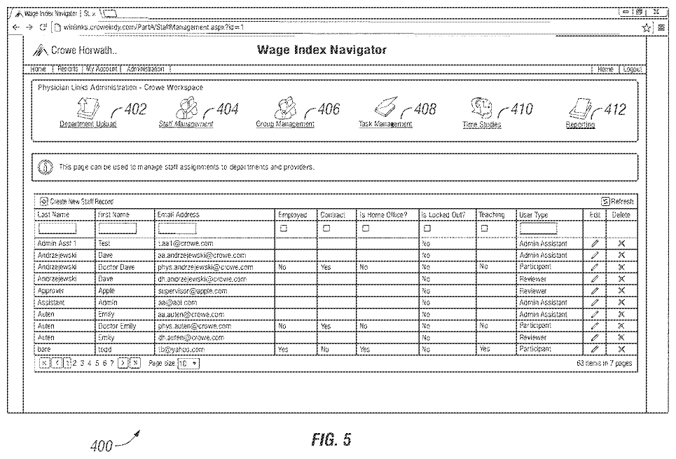

FIG. 5 is an example showing the user having selected the staff management category 404. This area of the system may be used to manage staff assignments to departments and providers. For example, new staff records may be created, edited, or staff records may be deleted. The customized settings for each staff member setup in the administrative module are tied to the user's account when logged in to the time entry module. By way of example only, the staff member can be associated with a subset of tasks from the entire universe of tasks that could be selected, which is customized specific for that user. In the administrative module, the user is associated in a subset of tasks; in the time entry module, the user will only be able to select from the subset of tasks associated with that user based on the setup in the administrative module. By reducing the tasks (or other options) that can be selected, this reduces the risk of errors during time entry.

FIG. 6 shows an example interface that may be used if the user selects to create new staff record in the interface of FIG. 5. This allows the user to enter information about the staff member to be added to the system. In the example shown, the user may step through the following types of information to be entered about a new staff record: staff management 600, account management 602, hospital assignment 604, department assignment 606, reviewer 608, and contract management 610. After entering information about the new staff member on this screen, the user may select the save button 612.

FIG. 7 is a screenshot if the user has entered information and selected the save button 610 in FIG. 6. In the example shown, the user may select the user type for the new record 700, whether the account should be active or inactive 702 and a password associated with the record 704. Once the user has entered the desired information, the save button 706 may be selected.

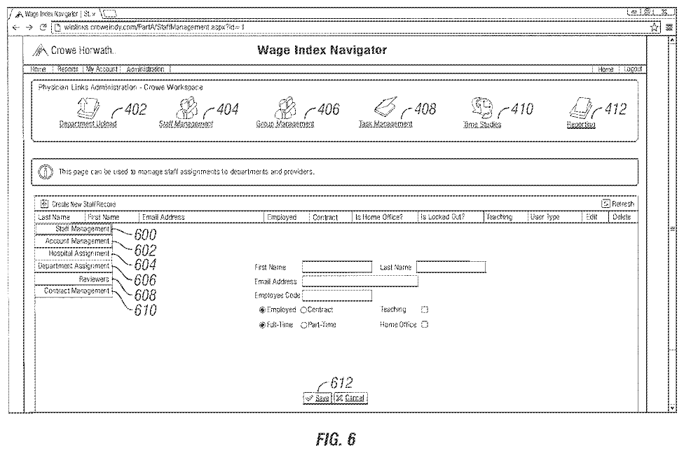

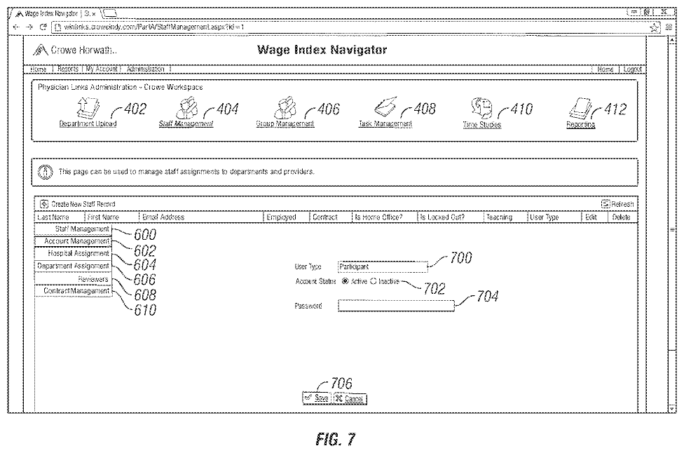

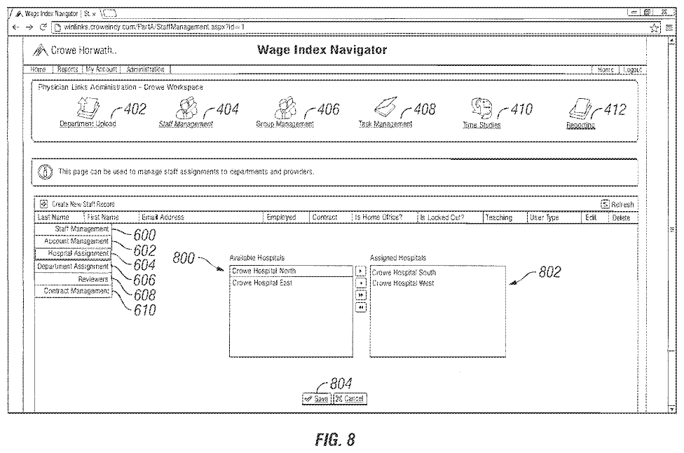

FIG. 8 shows an example interface to allow a hospital assignment 604 to be associated with the new record. In this example, the user may select from a plurality of available hospitals 800 and determine which of these should be considered hospitals assigned to the record 802. Once the desired hospitals are assigned, the user may select the save button 804.

FIG. 9 is an example screenshot showing an interface for department assignment 606. In this example, the user may select a particular hospital with which to associate departments for the new record 900. In some cases, if there are a large number of hospitals to search, the user may use a search interface 902 to filter the number of hospitals to select the desired hospitals. The user may then select from available departments 904 and assign a department to the record 906. Once the desired departments are assigned, the user may select the save button 908.

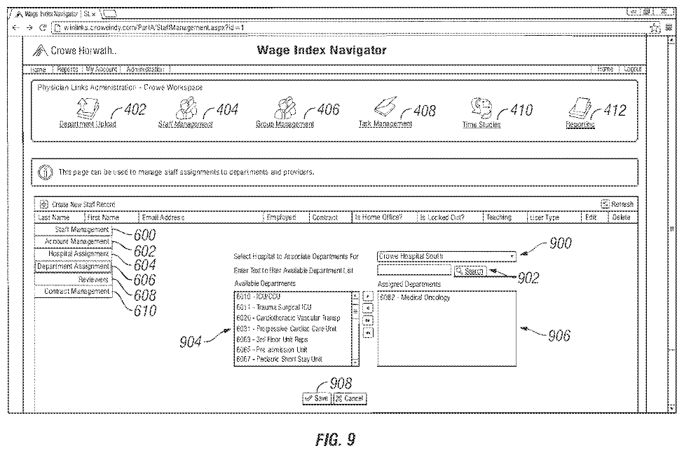

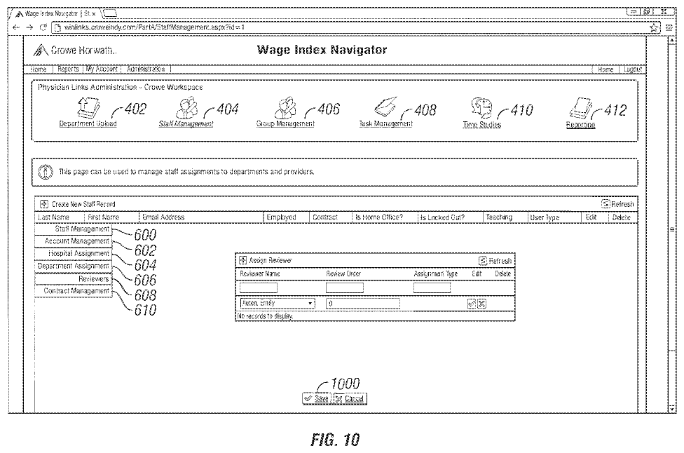

FIG. 10 shows an example interface that may be used to assign reviewers to the new record 608. These users may be forwarded time entries, for example, to approve or disapprove. Once the user has selected the desired reviewers, the user may select the save button 1000.

FIG. 11 shows an example interface that may be used for contract management 610. In this example, the user is allowed to enter identifying information about the contract 1100 along with parameters of the contract 1102. A digital copy of the contract may be uploaded to the system by using the upload interface 1104. Once the user has entered the desired information, the add button 1106 may be selected.

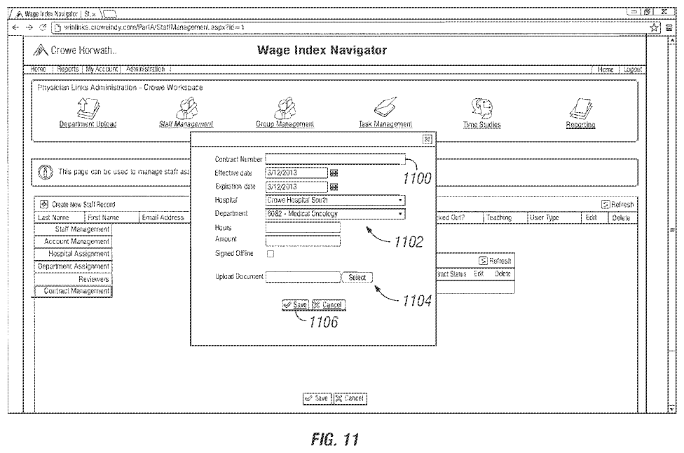

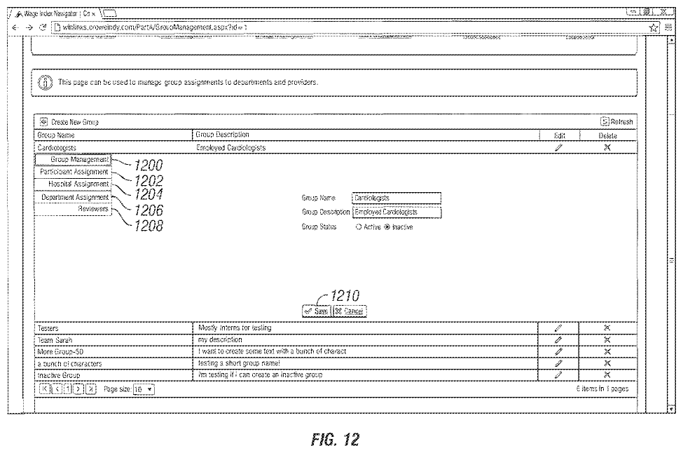

FIG. 12 shows an example where the user has selected the group management category 406. In this example, the user may select among a plurality of functions regarding management of groups; group management 1200, participant assignment 1202, hospital assignment 1204, department assignment 1206, and reviewers 1208. As shown in FIG. 12, a new group name may be added with a description and group status. Once the user has entered the desired information, the save button 1210 may be selected.

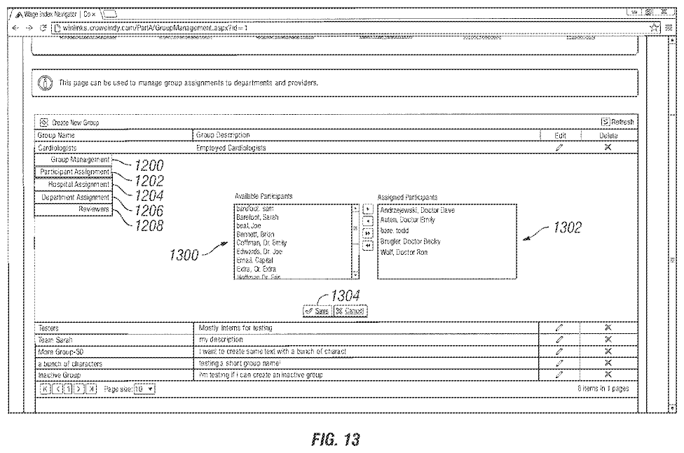

FIG. 13 shows an example interface that may be used for participant assignment 1202. In this example, there are a plurality of participants 1300 from which a user may select to be assigned participants 1302. Once the desired available participants have been assigned, the user may select the save button 1304.

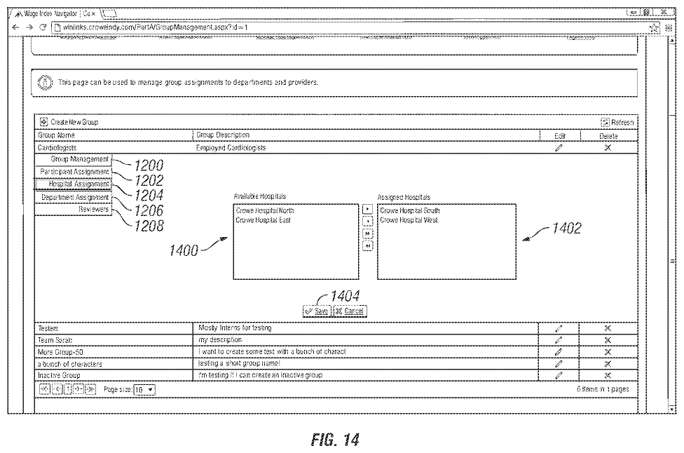

FIG. 14 shows an example interface for performing hospital assignments 1204. In this example, the user is presented with a plurality of available hospitals 1400 that may be selected for assignment of the record to particular hospitals 1402. Once the desired hospitals have been assigned, the user may select the save button 1404 to store the record.

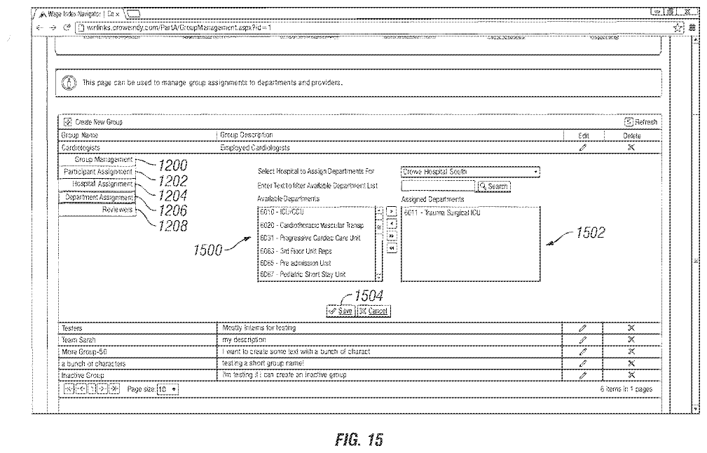

FIG. 15 shows an example interface that may be used to perform department assignments. In this example, the user is presented with a plurality of available departments 1500 from which to associate with assigned departments 1502. Once the user has selected the desired departments to assign to the record, the save button 1504 may be selected to store the record.

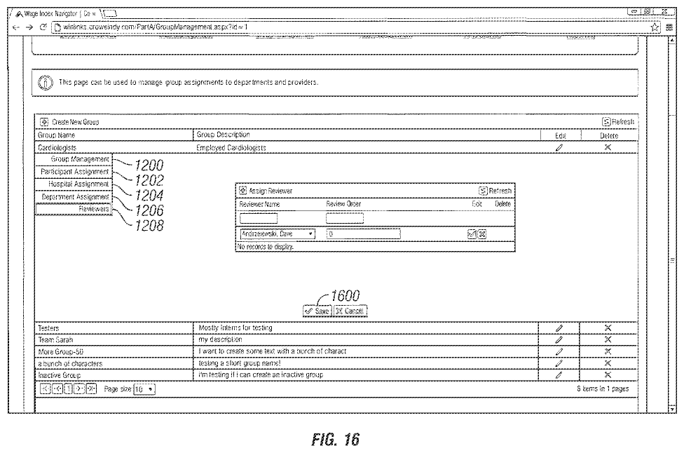

FIG. 16 shows an example interface that may be used to select reviewers 1208 associated with the record. Once the user has selected the desired reviewers for the record, the user may select the save button 1600 to store the record.

FIG. 17 shows an example interface after the user has selected the task management category 408. In the example shown, the user may create one or more new tasks to be stored in the database. In this example, the user may select a task name 1700 and a task type 1702.

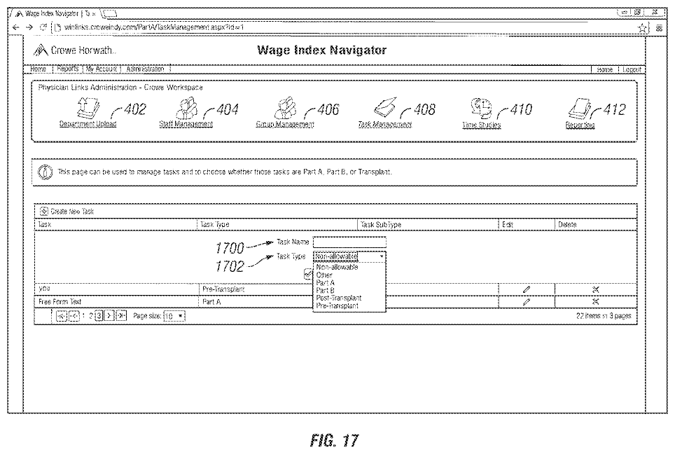

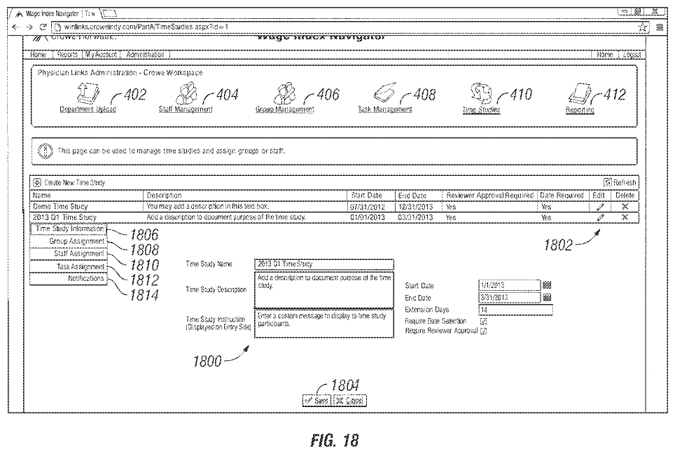

FIG. 18 shows an example interface upon the user selecting the time studies category 410. In the example shown, the user may create new time study records by entering data into an interface 1800, or may edit 1802 existing records. In the example shown, the user may create/edit the following types of information regarding a time study: time study information 1806, group assignment 1808, staff assignment 1810, task assignment 1812, and notifications 1814. Once the user has entered the desired information, the save button 1804 may be selected.

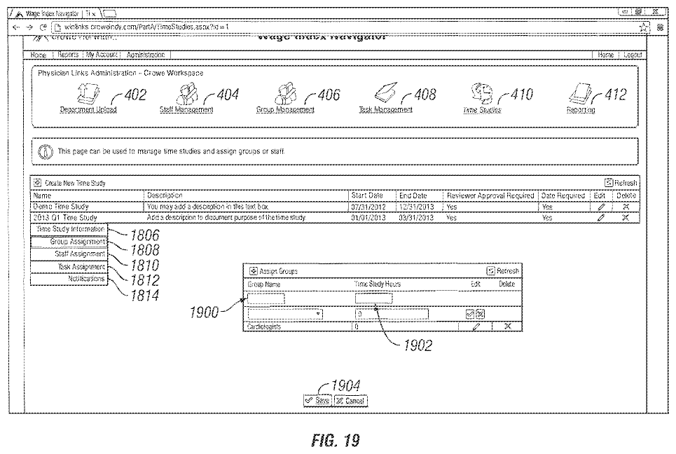

FIG. 19 shows an example interface that could be used for group assignment 1808. In this example, the user is presented with an interface allowing entry of a group name 1900 and time study hours 1902. Once the user has entered the desired information, the save button 1904 may be selected.

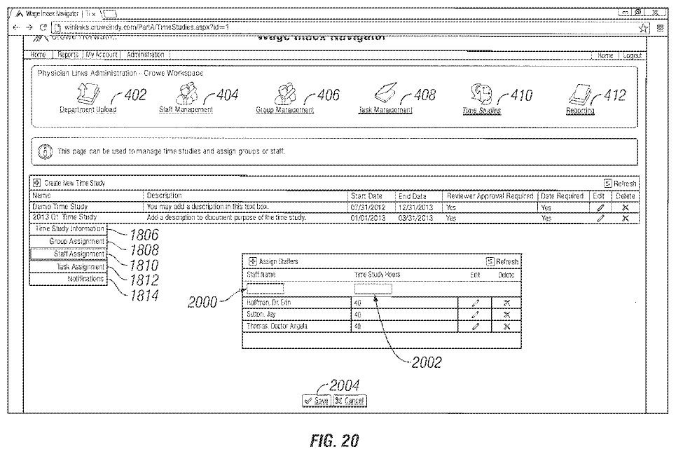

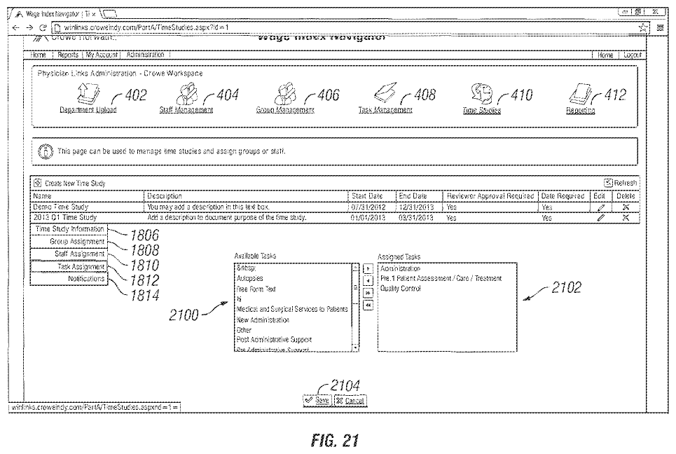

FIG. 20 shows an example interface that may be used for staff assignment 1810. In the example interface, the user is presented with an interface for selecting or entering a staff name 2000 and assign a time study hours value 2002. Once the user has entered the desired information, the save button 2004 may be selected to store the record.

FIG. 21 shows an example interface that may be used for task assignment 1812. In the example shown, the user is presented with a plurality of available tasks 2100 that may be assigned task 2102 for a study. Once the user has entered the desired information, the save button 2104 may be selected.

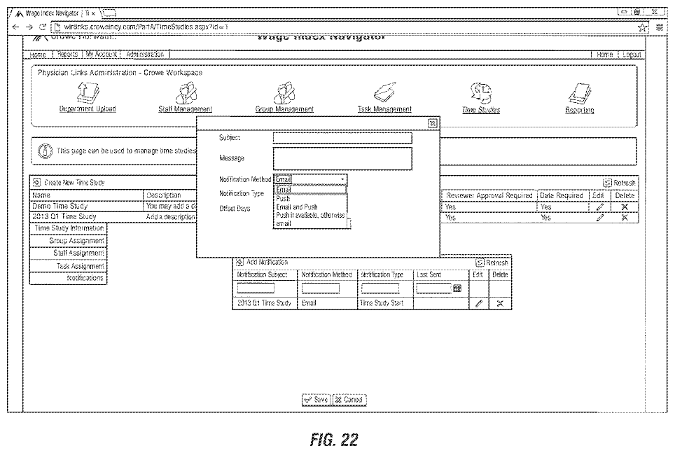

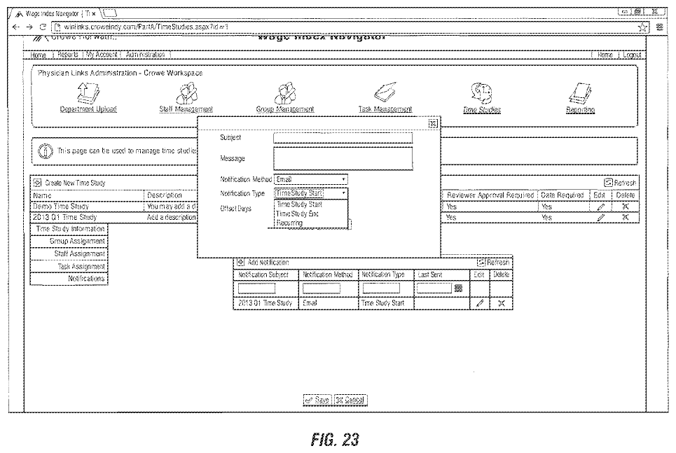

FIG. 22 shows an example interface for the notifications category 1814. In some cases, the system may provide notifications to the user to do one or more tasks, such as enter time or review time. In this example interface, the user may set up the appropriate type of notification for this record, whether it be email, push, email and push, or other type of notification. FIG. 23 is an example of another interface for setting up a notification regarding a time study.

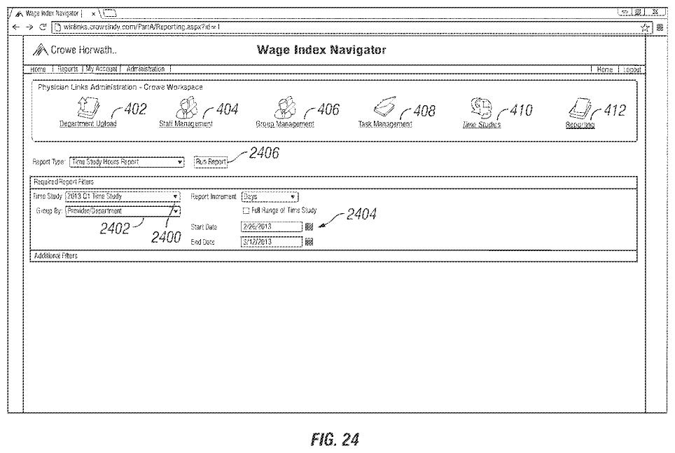

FIG. 24 shows an example interface that may be used upon the user selecting the reporting category 412. In this example, the user is presented with an interface that allows selection of a time study 2400 and an ability to format the report, such as being grouped by certain parameters 2402. The user may also select the time range 2404 for the time study. Once the user has configured the formatting and other entries for the report, the user may select the run report button 2406 to generate the desired report.

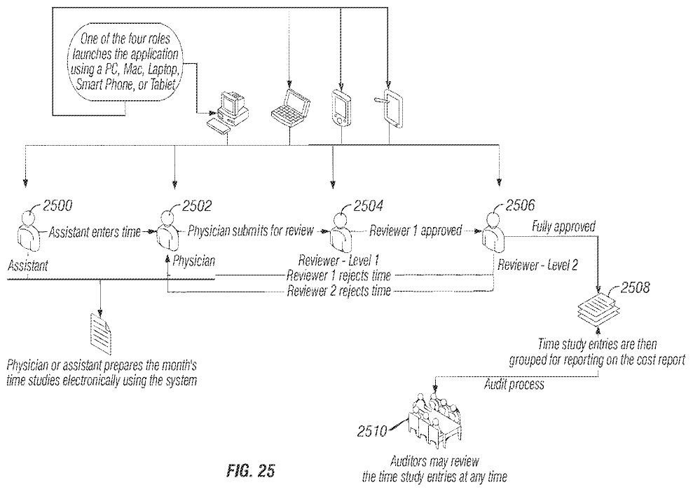

FIG. 25 is a diagrammatic view of an example time entry and review workflow, according to one embodiment. In the example shown, the workflow is role-based with an assistant 2500, a physician 2502, a first reviewer 2504, and a second reviewer 2506. In some cases, the physician 2502 may directly enter his/her own time and submit for review. In other cases, the assistant 2500 of the physician 2502 may enter the physician's time which is then provided to the physician 2502 for review prior to submitting for further review. In this example, there are two layers of review 2504, 2506 that could reject time prior to approval. For example, the first reviewer 2504 may be a department chair and the second reviewer 2506 may be the hospital administrator. Once a time study has been approved, this is stored in the system for reporting 2508. In the event the time study is audited, the auditors 2510 could review the documents associated with the time study stored in the system.

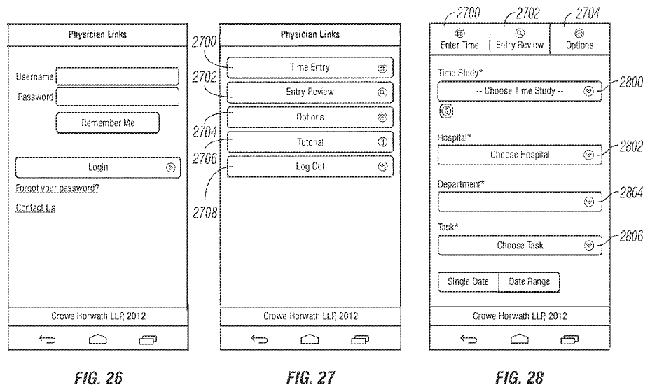

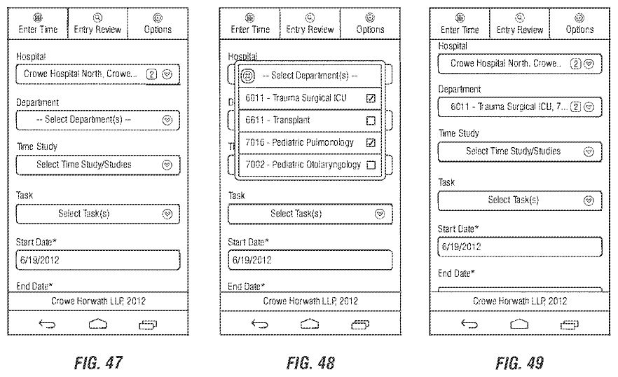

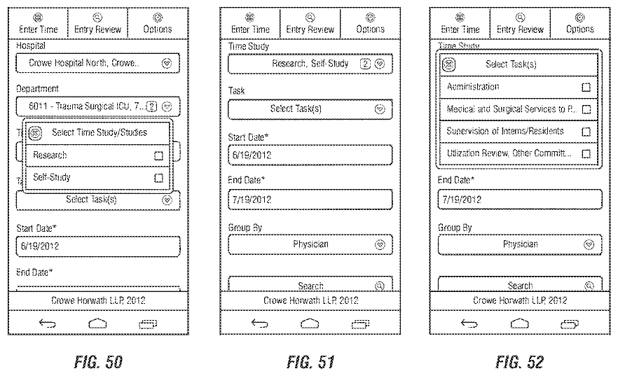

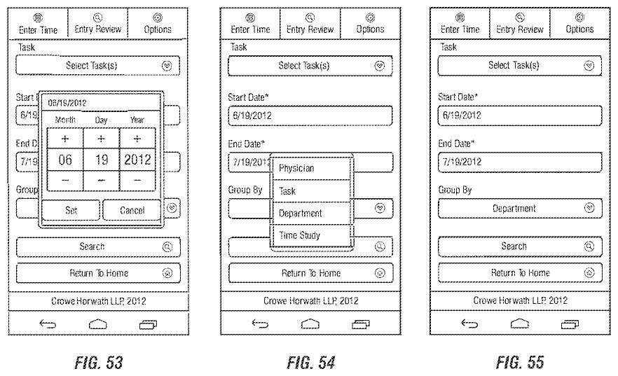

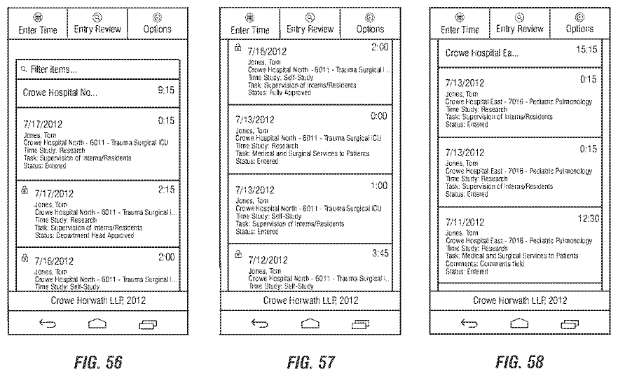

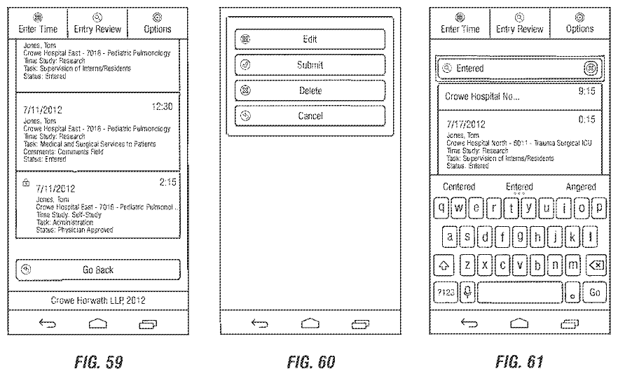

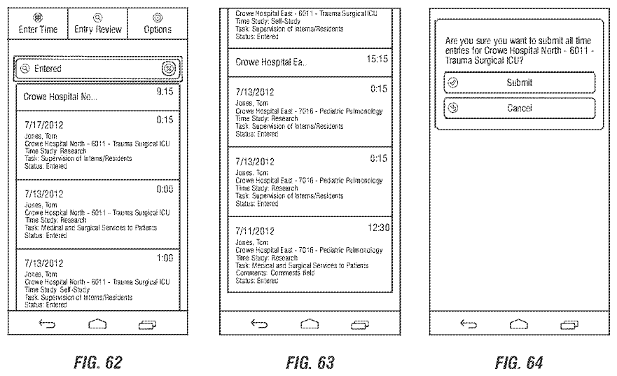

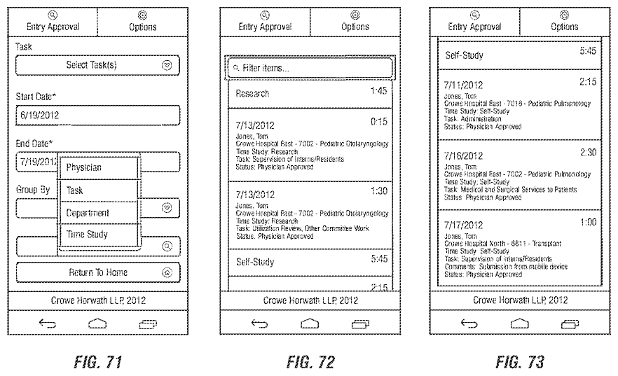

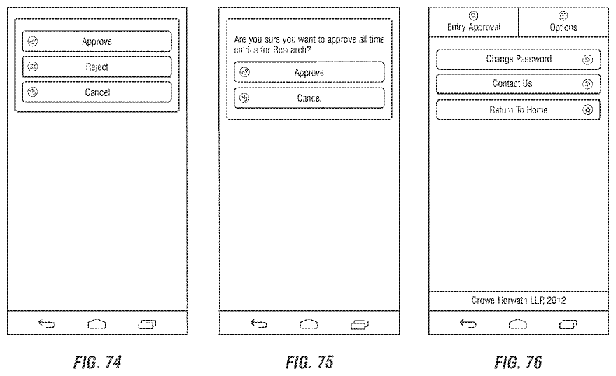

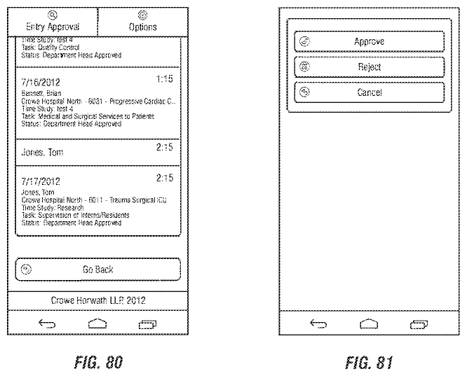

FIGS. 26-81 are screen shots showing possible functionality of a time entry/review module.

FIG. 26 shows an example login screen that may be used by an assistant 2500, a physician 2502, or a reviewer 2504, 2506 to access the system. In this example, the user must supply a user name and password to log into the system.

FIG. 27 shows a home screen upon the physician 2505 logging into the system. In this example, the user may perform a number of functions including: time entry 2700, entry review 2702, options 2704, tutorial 2706, and log out 2708.

FIG. 28 shows an example interface that may be used upon the user selecting time entry 2700. In this example, the user may select a time study 2800 from a plurality of time studies associated with the physician, along with other information, such as hospital 2802, department 2804, task 2806, and whether the entry will relate to a single date or date range.

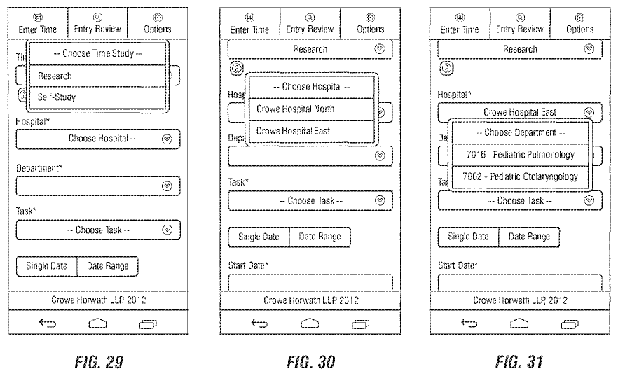

FIG. 29 shows the example interface of FIG. 28 in which the user has selected the time study pull down list 2800.

FIG. 30 shows the example interface of FIG. 28 in which the user has selected from the hospital pull down list 2802.

FIG. 31 shows the example interface of FIG. 28 in which the user has selected the department pull down list 2804.

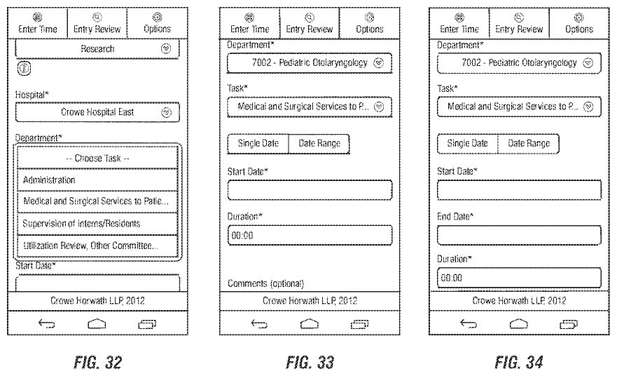

FIG. 32 shows the interface of FIG. 28 in which the user has selected the task pull down list 2806.

FIGS. 33 and 34 show example interfaces from which the user may enter time for a single date or a date range, respectively.

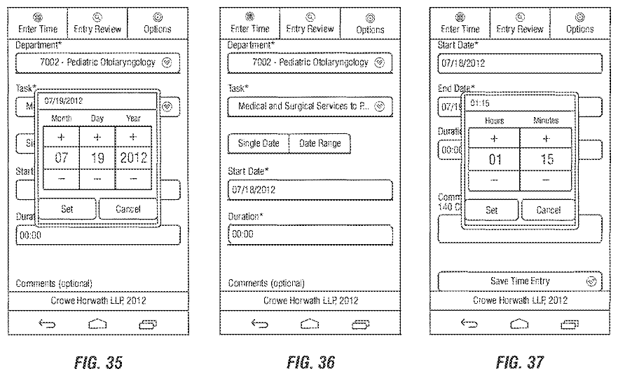

FIGS. 35-37 show an example interface for the user entering time information regarding a time entry.

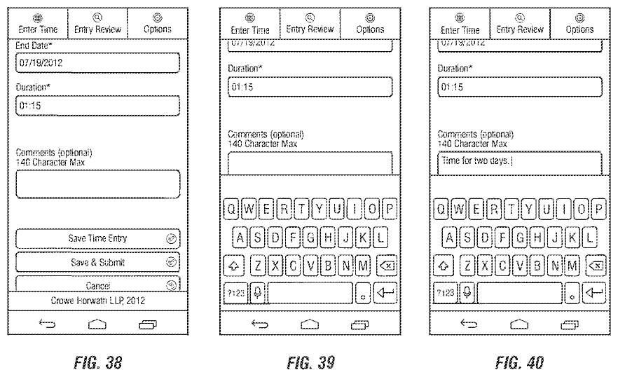

FIGS. 38-40 show an example interface in which the user may enter comments about the time entry.

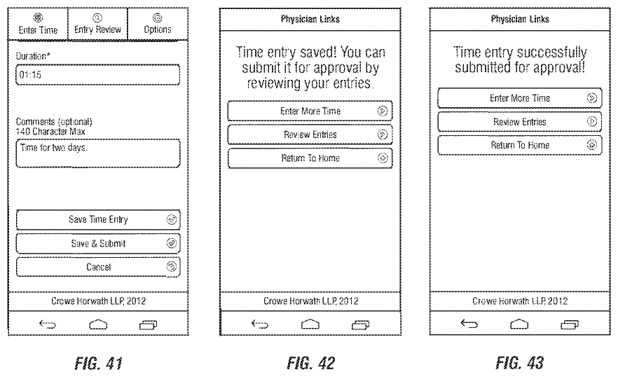

FIG. 41 shows an example in which the user may select to save the time entry, save and submit the time entry, or cancel the time entry.

FIG. 42 shows an example interface that may be used after the user has selected the save time entry option of FIG. 41. This allows the user to enter more time, review entries, or return to the home screen.

FIG. 43 shows an example interface that may be provided if the user selects the save and submit button on FIG. 41, which sends the entry to the first reviewer 2504 for review.

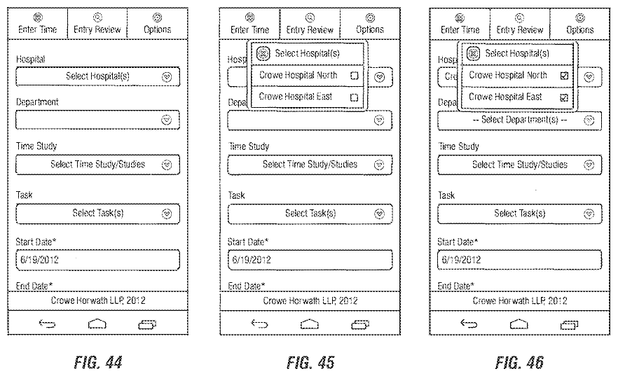

FIGS. 44-64 show an example interface that may be used by a user, such as a physician, to review entries entered by an assistant 2500. As can be seen, the user may select entries to be reviewed in a wide variety of manners and once approved, may submit the entries to the first reviewer 2504.

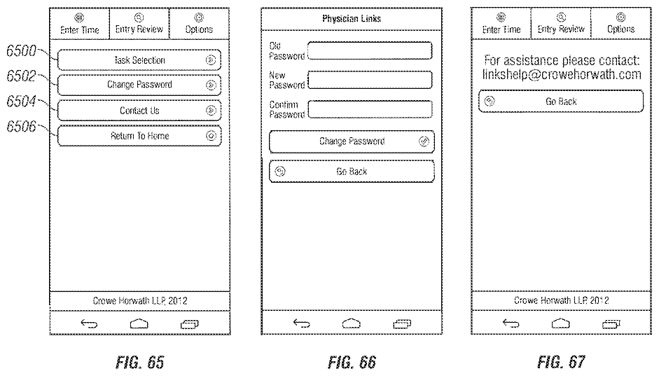

FIG. 65 shows an example in which the user has selected the options tab. In this example, the user may select from a variety of different options, such as task selection 6500, change password 6502, contact us 6504, or return to the home screen 6506.

FIG. 66 shows an example interface for changing the password of the user, upon selection of the change password button 6502.

FIG. 67 shows example contact information that may be displayed upon the user selecting the contact us button 6804.

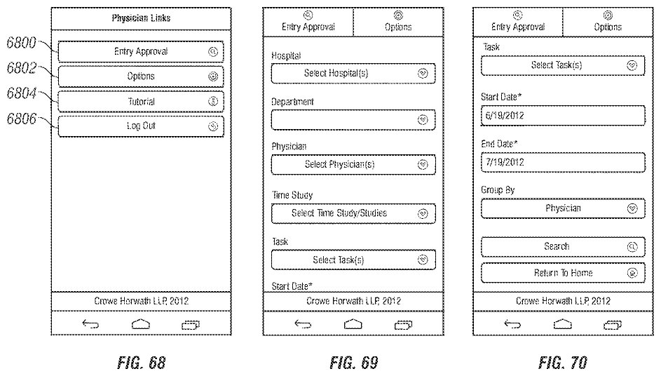

FIG. 68 shows an example home screen that may be used by a first reviewer 2504, such as a department head. In this example, the reviewer has a variety of options from which to select; entry approval 6800, options 6802, tutorial 6804, or logout 6806.

FIGS. 69-75 show an example interface that may be used to review and accept or reject time entries.

FIG. 76 shows an example interface that may be used if the user selects the options tab.

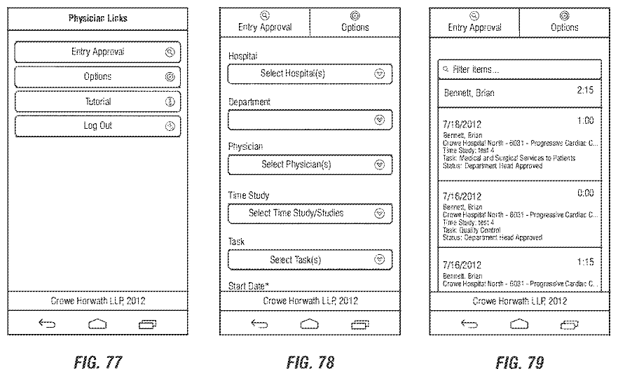

FIG. 77 shows an example home screen that may be used by a second reviewer 2506, such as a hospital administrator. FIGS. 78-81 show a variety of options to review and accept or reject time entries.

Although the present disclosure has been described with reference to particular means, materials, and embodiments, from the foregoing description, one skilled in the art can easily ascertain the essential characteristics of the invention and various changes and modifications may be made to adapt the various uses and characteristics without departing from the spirit and scope of the invention.

What is claimed is:

1. A computerized system of tracking time of health care providers, the system comprising:
an admin module to:
(i) assign a plurality of time entry requirements to a first time study, a second time study, and a third time study, wherein the time entry requirements for the first time study comprise a plurality of first acceptable task categories, the time entry requirements for the second time study comprise a plurality of second acceptable task categories, and the time entry requirements for the third time study comprise a plurality of third acceptable task categories, and wherein at least a portion of the first acceptable task categories, the second acceptable task categories, and the third acceptable task categories are different from each other;
(ii) assign a plurality of first health care providers, including a first user, but not a second user and not a third user, to the first time study;
(iii) assign a plurality of second health care providers, including the first user and the second user, but not the third user, to the second time study;
(iv) assign a plurality of third health care providers, including the second user and a third user but not including the first user, to the third time study;
a time entry module to:
(i) receive login credentials;
(ii) determine whether the login credentials are associated with the first user, the second user or the third user;
(iii) responsive to determining the login credentials are associated with the first user:
(a) present an interface from which the first user can select from a plurality of available time studies assigned to the first user, including the first time study and the second time study;
(b) receive a selection from the user interface of the first time study from the plurality of available time studies assigned to the first user;
(c) responsive to selection of the first time study, generate on the interface a portion from which the first user can record time entries for the first time study, wherein time entries include at least: (1) a time amount; and (2) a selected time category associated with the time amount, wherein the interface is configured to restrict selection of the selected time category for the first time study to the first acceptable task categories; and
(d) store the time entries for the first time study recorded by the first user;
(iv) responsive to determining the login credentials are associated with the second user:
(a) present an interface from which the second user can select from a plurality of available time studies assigned to the second user, including the second time study and the third time study;
(b) receive a selection from the user interface of the second time study from the plurality of available time studies assigned to the second user;
(c) responsive to selection of the second time study, generate on the interface a portion from which the second user can record time entries for the second time study, wherein time entries include at least: (1) a time amount; and (2) a selected time category associated with the time amount, wherein the interface is configured to restrict selection of the selected time category for the second time study to the second acceptable task categories; and
(d) store the time entries for the second time study recorded by the second user;
(v) responsive to determining the login credentials are associated with the third user,
(a) present an interface from which the third user can select from a plurality of available time studies assigned to the third user, including the third time study;
(b) receive a selection from the user interface of the third time study from the plurality of available time studies assigned to the third user;
(c) responsive to selection of the third time study, generate on the interface a portion from which the third user can record time entries for the third time study, wherein time entries include at least: (1) a time amount; and (2) a selected time category associated with the time amount, wherein the interface is configured to restrict selection of the selected time category for the third time study to the third acceptable task categories; and
(d) store the time entries for the third time study recorded by the third user.

2. The system of claim 1, wherein when the login credentials are determined to be associated with the first user, the interface prevents the first user from selection of the third time study.

3. The system of claim 2, wherein when the login credentials are determined to be associated with the second user, the interface prevents the second user from selection of the first time study.

4. The system of claim 3, wherein when the login credentials are determined to be associated with the third user, the interface prevents the third user from selection of both first time study and the second time study.

5. The system of claim 1, wherein the admin module includes an assignment interface to assign the plurality of first health care providers to the first time study, assign the plurality of second health care providers to the second time study, and assign the plurality of third health care providers to the third time study, wherein the assignment interface includes a group management portion configured to add and delete one or more health care providers to the first time study, the second time study, and/or the third time study.

6. The system of claim 5, wherein the assignment interface includes a group assignment portion in which assignments of health care providers to the first time study, the second time study, and/or the third time study are assigned by a group of multiple health care providers associated with a hospital and/or a hospital department associated with the health care providers.

7. The system of claim 1, wherein the admin module includes a time study creation interface to create and/or modify one or more of the first time study, the second time study, and/or the third time study.

8. The system of claim 7, wherein the time study creation interface is configured to enter one or more time entry requirements.

9. The system of claim 8, wherein the time study creation interface includes a portion with a plurality of available task categories for user-selection to associate with at least one time study.

10. The system of claim 1, wherein the admin module is further to assign one or more reviewers to each of the time studies and the time entry module further comprises to request approval of the captured time entries from the one or more reviewers assigned to the selected time study.
